(19) United States
(12) Patent Application Publication
(10) Pub. No.: US 2023/0010871 A1
(43) Pub. Date: Jan. 12, 2023

(54) COMPLEXES COMPRISING A CARBOHYDRATE POLYMER AND AN ACTIVE INGREDIENT AND PROCESSES FOR THEIR PREPARATION

(71) Applicant: FOLIUM LABS INC., Montreal, Québec (CA)

(72) Inventors: Felix POLYAK, Montreal, Québec (CA); Dmitri BOUDOVITCH, Montreal, Québec (CA)

(21) Appl. No.: 17/784,468

(22) PCT Filed: Dec. 11, 2020
(86) PCT No.: PCT/CA2020/051713

§ 371 Date: Jun. 10, 2022

Related U.S. Application Data
(60) Provisional application No. 62/947,919, filed on Dec. 13, 2019.
(60) Provisional application No. 63/060,360, filed on Aug. 3, 2020.

(51) Int. Cl. A61K 47/61; A61K 41/00; A23L 33/105; A23L 33/125; A23L 2/52

(52) U.S. Cl. CPC A61K 47/61; A61K 41/00; A23L 33/105; A23L 33/125; A23L 2/52; A23V 2002/00

(57) ABSTRACT

Molecular complexes and compositions containing the same are disclosed herein. More specifically, carbohydrate polymers, for example hyaluronic acid or a salt thereof, are complexed with a biologically active compound selected from natural products and nutrients (amino acids, amino esters, hydroxy acids, hydroxy esters, vitamins, cannabinoids, etc.), and active pharmaceutical ingredients to create stabilized molecular complexes. The complexation can be conveniently achieved by means of a resonant acoustic mixing process.

### RELATED APPLICATIONS

This application claims priority under applicable laws to U.S. provisional application No. 62/947,919 filed on Dec. 13, 2019, and to U.S. provisional application No. 63/060,360 filed on Aug. 3, 2020, the contents of which is incorporated herein by reference in their entirety for all purposes.

### TECHNICAL FIELD

The present technology generally relates to compositions created from carbohydrate polymers and organic molecules. More specifically, but not exclusively, the present disclosure relates to molecular complexes of carbohydrate polymers with biologically active molecules. The present disclosure also relates to a process for the production of the mentioned above compositions comprising resonant acoustic mixing.

### BACKGROUND

Carbohydrate polymers are a class of naturally occurring polymers in various forms, which may be used in a variety of fields. Hyaluronic acid (or a salt thereof, collectively referred to as HA) is one example of a naturally occurring carbohydrate polymer. HA is a polyanionic, non-sulfated glycosaminoglycan that consists of N-acetyl-D-glucosamine and β-glucuronic acid. It is present in the intercellular matrix of most vertebrate connective tissues especially skin and joints where it has a protective, structure stabilizing and shock-absorbing role.

The unique viscoelastic nature of HA along with its biocompatibility and non-immunogenicity has led to its use in a number of clinical applications, which include: the supplementation of joint fluid in arthritis; as a surgical aid in eye surgery; and to facilitate the healing and regeneration of surgical wounds. More recently, HA has been investigated as a drug delivery agent for various routes of administration, including ophthalmic, nasal, pulmonary, parenteral and topical, for instance, as a simple mixture of the drug and HA, or as a drug crosslinked on the HA polymer.

Complexes of HA and small organic molecules have been prepared in the past, for instance, using intensive mechanical and mechano-chemical methods, which include mixing and applying pressure and shear deformation. Such methods are applied to devices such as ball mill, Bridgman anvil, extruder, etc. However, these methods have some drawbacks such as low reproducibility, low control of the reaction product, particle size distribution, required pre-mixing which adds a step to the method, limitation to small scale, high cost, and/or low reliability. These are at the first glance, other could be provided after certain analysis.

Accordingly, there is a need for new processes of producing new active molecule delivery composition having improved properties, for instance, improved bioavailability, targeted delivery, or other improved properties compared to existing technologies.

### SUMMARY

The invention described herein generally relates to processes for manufacturing complexes comprising carbohydrate polymers and biologically active ingredients, which comprises a resonant acoustic mixing step, a physical method, and to the complexes produced therefrom, their compositions and uses. Also described are complexes comprising a carbohydrate polymer and a biologically active ingredient regardless of its method of making. Edible (food, beverages, etc.) compositions comprising the present complexes are also contemplated.

According to one aspect, the present technology relates to a process for preparing a complex of a carbohydrate polymer and at least one biologically active compound, the process comprising:
(a) mixing the carbohydrate polymer with the at least one biologically active compound to produce a mixture;
(b) feeding the mixture into a resonant acoustic mixer (RAM); and
(c) operating the RAM under resonant acoustic conditions to produce the complex.

For instance, the resonant conditions are carried out at moderate frequency (around 60 Hz) and forcing energy of between 50 g to 100 g. In one embodiment, step (c) is carried out at a temperature of between about 20° C. and about 40° C., or between about 25° C. and about 30° C. In another embodiment, step (c) is carried out at a frequency of about 20 Hz to about 90 Hz. In a further embodiment, step (c) comprises a residence time of about 10 second to about 10 minutes, or between about 30 seconds and 5 minutes.

According to another aspect, the present technology relates to a process for preparing a complex of a carbohydrate polymer and at least one biologically active compound, the process comprising:
a) adding the carbohydrate polymer and the at least one biologically active compound into a RAM; and
b) mixing the carbohydrate polymer and the at least one biologically active compound under resonant acoustic conditions at moderate frequency (around 60 Hz) and forcing energy of between 50 g to 100 g to produce the complex.

According to one embodiment, step (b) is carried out at a temperature of between about 20° C. and about 40° C., or between about 25° C. and about 30° C. In another embodiment, step (b) is carried out at a frequency of about 20 Hz to about 90 Hz. In a further embodiment, step (b) comprises a residence time of about 10 second to about 10 minutes, or between about 30 seconds and 5 minutes.

According to one embodiment of any of the above-described processes, the carbohydrate polymer is selected from glycosaminoglycans (e.g. hyaluronic acid or its salts), cellulose, starch (amylose, amylopectin), chitin, chitosan, inulin, cyclodextrin, and the like, for instance, the carbohydrate polymer is hyaluronic acid or a salt thereof.

In one embodiment, the complex comprises from about 50 to about 95% (w/w) of the carbohydrate polymer. In another embodiment, the complex comprises from about 5 to about 50% (w/w) of the biologically active compound. In a further embodiment, the weight ratio of biologically active compound to carbohydrate polymer is from 1:1 to 1:100, or from 1:4 to 1:9.

In other embodiment, the biologically active compound is selected from amino acids, amino esters, amino alcohols, hydroxy acids, hydroxy esters, vitamins, and combinations thereof.

According to one embodiment, the vitamin is selected from vitamin A, vitamin B1, vitamin B2, vitamin B3, vitamin B5, vitamin B6, vitamin B9, vitamin B12, vitamin C, vitamin D, vitamin E, vitamin H, vitamin K, and combinations thereof.

In a further embodiment, the biologically active compound is a pharmaceutical active ingredient selected from small molecule drugs, including antineoplastic agents, antibiotics, antivirals, analgesics, anticoagulants, antidepressants, psychedelic therapy drugs (for example for OCD, PTSD, alcoholism, depression, cluster headaches, etc. including psilocybin, LSD, DMT, ketamine, etc.), antipsychotics, sedatives, anti-inflammatory agents, antidiabetics, cardiovascular agents, and the like. In one embodiment, the biologically active compound has a molecular weight below 0.9 kDa.

In yet another embodiment, the biologically active compound has a molecular weight of at least 0.9 kDa. In an embodiment, the biologically active compound is a pharmaceutical active ingredient selected from nucleic acids, proteins and peptides, for instance a monoclonal antibody.

In other embodiments, the biologically active compound is a natural product or extract. In an embodiment, the natural product or extract comprises turmeric (or curcumin) or a cannabinoid, for instance, selected from tetrahydrocannabinol, cannabidiol, cannabinol, cannabigerol, cannabichromene, cannabicyclol, cannabivarin, tetrahydrocannabivarin, cannabidivarin, cannabichromevarin, cannabigerovarin, cannabigerolic acid, delta-tetrahydrocannabinolic acid, cannabidiolic acid, cannabichromenenic acid, cannabigerovarinic acid, tetrahydrocannabivarinic acid, cannabidivarinic acid, cannabichromevarinic acid, and the like.

According to another aspect, the present technology relates to a complex prepared by a process as defined herein or a complex of a carbohydrate polymer and at least one biologically active compound. In one embodiment, the carbohydrate polymer is as defined above. In another embodiment, the biologically active compound is as defined above.

In one embodiment, the complex comprises from about 50 to about 95% (w/w) of the carbohydrate polymer. In another embodiment, the complex comprises from about 5 to about 50% (w/w) of the biologically active compound. In a further embodiment, the weight ratio of biologically active compound to carbohydrate polymer is from 1:1 to 1:100, or from 1:4 to 1:9.

According to a further aspect, the present technology relates to a composition comprising the complex as defined herein together with a carrier. In one embodiment, the composition further comprises a stabilizer. In one embodiment, the composition is a solid composition (e.g. powder composition). In another embodiment, the composition is a liquid composition. In an further embodiment, wherein the concentration of the complex in the composition is from about 0.6 to about 10% (w/w). According to another embodiment, the composition is a pharmaceutical composition for use as a medication. In another embodiment, the pharmaceutical composition being in the form of a tablet (e.g. hard-pressed or chewable tablet) or gel capsule.

According to a further aspect, the present technology also relates to an edible composition comprising the complex as defined herein together with a carrier. In one embodiment, the carrier is selected from water or other aqueous based drinkable solutions like dealcoholized beer or wine, tea, natural or artificial juice, gelatin, dough, chocolate, etc. In another embodiment, the edible composition is a solid or semi-solid food composition (e.g. cookies, cakes and other pastries, puddings, chocolates, soft and hard candies (such as gummies and mints)). In another embodiment, the edible composition is a liquid composition (e.g. beverages (such as water, juices, soft drinks, tea, dealcoholized wine or beer, etc.) or concentrated beverages).

In a preferred embodiment, the biologically active compound in the edible composition is selected from a natural product or extract comprising a nutraceutical or a cannabinoid, and a psychedelic therapy drug (e.g. psilocybin, LSD, DMT, ketamine, etc.). For instance, the natural product or extract comprises turmeric (or curcumin) or a cannabinoid, such as tetrahydrocannabinol, cannabidiol, cannabinol, cannabigerol, cannabichromene, cannabicyclol, cannabivarin, tetrahydrocannabivarin, cannabidivarin, cannabichromevarin, cannabigerovarin, cannabigerolic acid, delta-tetrahydrocannabinolic acid, cannabidiolic acid, cannabichromenenic acid, cannabigerovarinic acid, tetrahydrocannabivarinic acid, cannabidivarinic acid, cannabichromevarinic acid, and the like.

In a preferred embodiment, the carbohydrate polymer in the complex is a glycosaminoglycan (e.g. hyaluronic acid or a salt thereof).

According to a further aspect, the present technology relates to the use of a complex as herein defined or of a composition as defined herein, in the treatment of neoplasm, bacterial infection, viral infection, pain, depression, sleep disorders, inflammation, diabetes, as a cardiovascular agent, anticoagulant or antipsychotic agent, or of diseases and disorders which could benefit from cannabinoid or psychedelic drug therapy.

### BRIEF DESCRIPTION OF THE FIGURES

FIG. 1 presents CBD plasma concentrations following administration at 20 mg/kg (CBD-based doses) to male Sprague Dawley rats. Data are presented as mean±SEM.

FIG. 2 shows the area under the curve (AUC) of CBD plasma concentrations following administration at 20 mg/kg (CBD-based doses) to male Sprague Dawley rats. Data are presented as mean±SEM. The * indicated P-value<0.05 (0.047) from a Student's t-test.

FIG. 3 presents THC plasma concentrations following administration at 20 mg/kg (THC-based doses) to male Sprague Dawley rats. Data are presented as mean±SEM.

FIG. 4 shows the AUC of THC plasma concentrations following administration at 20 mg/kg (THC-based doses) to male Sprague Dawley rats. Data are presented as mean±SEM. The ** indicated P-value<0.01 (0.0088) from a Student's t-test.

FIG. 5 presents letrozole plasma concentrations following administration at 2.5 mg/kg and 5.0 mg/kg (letrozole based doses) to rats.

### DETAILED DESCRIPTION

All technical and scientific terms and expressions used herein have the same definitions as those commonly understood by the person skilled in the art when relating to the present technology. The definition of some terms and expressions used herein is nevertheless provided below for clarity purposes.

1. Definitions

The word “a” or “an” when used in conjunction with the term “comprising” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this specification and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

When the term “approximately” or its equivalent term “about” are used herein, it means in the region of, and around. When the terms “approximately” or “about” are used in relation to a numerical value, it modifies it; for example, it could mean above and below its nominal value by a variation of at least 1%. This term may also take into account the probability of random errors in experimental measurements or rounding.

The term “resonant acoustic mixer” or “RAM”, as used herein, is intended to refer to any conventional device uses resonant acoustic mixing combining resonance and sound energy. For instance, resonant acoustic is a contactless mixing technology involving a combination of moderate frequency and relatively large displacement. For instance, manufacturers of RAMs describes the moderate frequency to be of about 60 Hz and the relatively large displacement to be of about 0.55″ for some of their mixers. Preferably, the resonant conditions are carried out at moderate frequency (around 60 Hz) and forcing energy of between 50 g to 100 g.

The term “complexed” or “complex” as used herein refers to the product of a process, in which at least two molecules, or two portions of a long molecule, are creating a complex on the molecular level by a non-chemical interaction (i.e. not by a covalent bond). Such interactions occur in many different ways including, for example, formation of a non-covalent bond, formation of hydrogen bonds, van der Waals, and/or hydrophobic, hydrophilic, ionic and/or electrostatic interactions. In further examples, molecular interactions are also characterized by an at least temporary physical connection between at least one molecule with itself or between two or more molecules.

The expression “carbohydrate polymer” or a similar expression as used herein designates a polymer comprising polymerized carbohydrate monomeric units, and including polysaccharide compounds, such as glycosaminoglycans (e.g. hyaluronic acid or a salt thereof), cellulose, starch (amylose, amylopectin), chitin, chitosan, inulin, cyclodextrin, and the like, and which are suitable for pharmaceutical use. The carbohydrate polymers equally include naturally occurring polymers and their synthetic equivalents, as well as their salts and derivatives (e.g. carboxymethyl cellulose, etc.).

As used herein, the expression “biologically active” refers to the ability to mediate a biological function. Similarly, the expression “biologically active compound” used herein refers to compounds that mediate a biological function and that comprise at least one group that participates to the formation of a complex as defined herein with the carbohydrate polymer. The expression excludes minerals. Examples of biologically active compounds include, without limitations, nutrients and nutraceuticals such as amino acids, amino esters, amino alcohols, hydroxy acids, hydroxy esters, vitamins, natural products and extracts (e.g. turmeric or curcumin), cannabinoids (e.g. tetrahydrocannabinol, cannabidiol, cannabinol, cannabigerol, cannabichromene, cannabicyclol, cannabivarin, tetrahydrocannabivarin, cannabidivarin, cannabichromevarin, cannabigerovarin, cannabigerolic acid, delta-tetrahydrocannabinolic acid, cannabidiolic acid, cannabichromenenic acid, cannabigerovarinic acid, tetrahydrocannabivarinic acid, cannabidivarinic acid, cannabichromevarinic acid, and the like), and combinations thereof, as well as small molecule drugs (e.g. with a molecular weight below 0.9 kDA or 900 g/mol), including antineoplastic agents (chemotherapy) and other oncology-related treatments (e.g. immunotherapies, adjuvants, etc.), antibiotics, antivirals, analgesics, anticoagulants, antidepressants, psychedelic therapy drugs (e.g. for OCD, PTSD, alcoholism, depression, cluster headaches, etc. including psilocybin, LSD, DMT, ketamine, etc.), antipsychotics, sedatives, anti-inflammatory agents, antidiabetics, cardiovascular agents, and the like, and larger active molecules or biologics (e.g. with a molecular weight above 0.9 kDA or 900 g/mol) such as nucleic acids, proteins and peptides (e.g. growth hormone proteins like somatropin, monoclonal antibodies, etc.).

The expressions “antineoplastic agents”, “antitumor agent”, and “anticancer agent” as used herein equally refer to agents used in the inhibition of cancer cell growth, reduction and/or elimination of cancer cells. Examples of antineoplastic agents include, without limitation, azacitidine, imatinib, lenalidomide, etoposide, topotecan, irinotecan, letrozole, raloxifene, cyclophosphamide, mechlorethamine, carbazylquinone, melphalan, thiotepa, busulfan, nimustine, carmustine, procarbazine, dacarbazine, methotrexate, 6-mercaptopurine, 6-thioguanine, azathioprine, 5-fluorouracil, ftorafur, floxuridine, cytarabine, ancitabine, doxifluridine, actinomycinD, bleomycin, mitomycin, chromomycin A3, cinelbin A, aclacinomycin A, adriamycin, peplomycin, cisplatin, mitoxantrone, epirubicin, pirarubicin, vinblastine, vincristine, vindesine, carboplatin, estramustine phosphate, mitotane, porphyrin, paclitaxel and docetaxel. Other examples include boron-containing compounds such as mercaptoundecahydrododecaborate (BSH) or β-boronophenylalanine (BPA), optionally used in boron neutron capture therapy (BNCT).

The term “amino acid” as used herein refers to any one of the following L—or D-amino acids: isoleucine, alanine, leucine, asparagine, lysine, aspartic acid, methionine, cysteine, phenylalanine, glutamic acid, threonine, glutamine, tryptophan, glycine, valine, proline, arginine, serine, histidine, and tyrosine, or a pharmaceutically acceptable salt thereof.

The term “amino ester” or “amino acid ester” as used herein refers to an alkyl, aryl, or arylalkyl ester formed on the carboxylic acid moiety of an amino acid. The definition of amino esters also encompasses their pharmaceutically acceptable salts.

The term “hydroxy acid” as used herein refers to an organic compound that is functionalized, at least, with both a hydroxyl group and a carboxylic acid group, or a pharmaceutically acceptable salt thereof. In an embodiment, the hydroxy acid is an alpha hydroxy acid, where the hydroxyl group is bonded to the carbon adjacent to the carboxylic acid group. Non-limiting examples of hydroxy acids include glycolic acid, malic acid, lactic acid, mandelic acid, phytic acid, salicylic acid, aleuritic acid, tartaric acid, citric acid, hydroxytetronic acid, glucuronic acid, mucic acid, galacturonic acid, gluconic acid, saccharic acid, glucoheptonic acid, alpha-hydroxybutyric acid, tartronic acid, alpha-hydroxyisobutyric acid, isocitric acid, alpha-hydroxyisocaproic acid, dihydroxymaleic acid, alpha-hydroxyisovaleric acid, dihydroxytartaric acid, beta-hydroxybutyric acid, dihydroxyfumaric acid, beta-phenyllactic acid, atrolactic acid, galactonic acid, pantoic acid and glyceric acid. Derivatives of the hydroxy acids are also encompassed by the definition. A preferred hydroxy acid is tartronic acid.

The term “hydroxy ester” as used herein refers to an alkyl, aryl, or arylalkyl ester formed on the carboxylic acid moiety of a hydroxy acid.

The term “vitamin” as used herein refers to any of the common nutrients required by an organism that are generally provided in an organism's diet and includes, for example, vitamins A, B₁, B₂, B₃, B₅, B₆, B₇, B₆, B₁₂, C, D, E, H and K. The term vitamin also encompasses derivatives of such vitamins. Non-limiting examples of vitamin derivatives include ascorbyl tetraisopalmitate, magnesium ascorbyl phosphate and ascorbyl glucoside, tocopheryl acetate, tocopheryl palmitate and tocopheryl linoleate.

The term “at least a portion” as used herein means that the entire amount of biologically active compound need not be complexed with the carbohydrate polymer, so long as a portion of the biologically active is complexed. For example, a portion may be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% or any range derivable therein.

The expression “group that participates to the formation of a complex” as used herein refers to any elements or combinations of structural elements which can contribute to the formation of a complex with the carbohydrate polymer. Non-limiting examples of such elements include oxygen, nitrogen or sulfur-containing groups such as hydroxy, ether, carbonyl, carboxy, ester, amino, amide, carbamate, urea, heterocycles (aromatic or non-aromatic), thiol, thioether, etc. Other groups may also be included.

As used herein, the term “stabilizing” includes maintaining a compound in a specific state and preventing or slowing fluctuations from that particular state into another.

As used herein, the terms “stabilizer,” or “preservative” include an agent that prevents the oxidation of other compounds. Examples of preservatives useful in the compositions of the present disclosure include, but are not limited to, an antioxidant, alpha-lipoic acid, 1-carnitine, phenoxyethanol, butylated hydroxytoluene and sodium benzoate. In an embodiment of the present disclosure, the antioxidant includes glutathione. One of ordinary skill in the art will appreciate that other preservatives are useful as additives in the present compositions. When a preservative is present, it may typically be present in an amount of from about 0.1% to about 1.5% by weight in the composition.

As used herein, the term “derivative” refers to a structural analog and designates a compound having a structure similar to that of another but differing from it with respect of a certain component or functional group. For instance, it can differ in one or more atoms, functional groups, or substructures, which are replaced with other atoms, groups, or substructures. A structural analog can be imagined to be formed, at least theoretically, from the other compound. Examples of derivatives include, without limitation, an ether or ester of a hydroxyl group, an ester or amide formed from carboxylic acid, an amide, carbamate, or urea of an amine group, and other similar groups.

As used herein, the term “prolonged action” refers to long acting formulations, that is, formulations that have pharmacokinetic characteristics such that the formulation provides for an extended length of release time than is normally found for the released drug itself.

II. The Processes

In one example, the present disclosure includes a process for preparing a complex of a carbohydrate polymer and at least one biologically active compound, the process comprising:
mixing the carbohydrate polymer with the at least one biologically active compound to produce a mixture; and
feeding the mixture into a resonant acoustic mixer (RAM); and
operating the RAM under resonant acoustic conditions to produce the complex.

In another example, the process for preparing a complex of a carbohydrate polymer and at least one biologically active compound comprises:
adding the carbohydrate polymer and the at least one biologically active compound into a RAM; an
mixing the carbohydrate polymer and the at least one biologically active compound under resonant acoustic conditions to produce the complex.

For instance, the mixture is prepared by thoroughly mixing the carbohydrate polymer with the at least one biologically active compound as defined herein without the addition of a solvent. The active ingredient, and any additional ingredient, may be briefly warmed up to 60° C. or 65° C. before mixing with the carbohydrate polymer. The mixture is then fed into the RAM, which is operated to result in a carbohydrate polymer that is complexed with the at least one biologically active compound.

The RAM apparatus is a mixer which provided a resonant acoustic treatment. The reaction takes place in a closed reaction vessel of the RAM apparatus of appropriate size, depending on the instrument model. For instance, manufacturers offer RAM equipments in various sizes. The reaction vessel may be made from glass, plastic or even metal, preferably glass or plastic. The method may be suitable for GMP (Good Manufacturing Practices) manufacturing. For instance, the mixture may be placed into the reaction vessel in a clean environment (e.g. clean room) and then placed into the RAM apparatus in a lesser clean room (the reaction vessel remaining closed). Opening of the reaction vessel after reaction could then be made back in a clean room.

The resonant acoustic mixing of the substantially homogeneous mixture can be carried out, for instance, a temperature of about 20° C. to about 40° C., or at about 25° C. to about 30° C. The frequency of the resonant acoustic mixing may be from about 20 Hz to about 90 Hz. Preferentially, the resonant conditions are carried out at moderate frequency (around 60 Hz) and forcing energy of between 50 g to 100 g. The resonant acoustic mixing is generally carried out for a period sufficient for the complex to form, for instance, between about 10 second to about 10 minutes, or about 30 seconds and 5 minutes, or between 30 seconds and 2 minutes.

In one example, the biologically active compound and carbohydrate polymer are weight out and placed into reaction vessel without pre-mixing. The weight ratio of biologically active compound and carbohydrate polymer may vary from 1:1 up to 1:100 (or between 1:4 to 1:9). The reaction mixture is then placed into the reaction chamber or vessel and parameters (time, temperature and frequency) are set as indicated above. For instance, the reaction is carried out at room temperature for less than 5 minutes. The reaction vessel is then taken out the RAM instrument and the complex formed is an amorphous powder, which is removed from the reaction vessel.

In some examples, the complex may comprise additional ingredients, excipients or active compounds, and which may be added to the biologically active compound or to the carbohydrate polymer before mixing or to the mixture in the RAM. In some examples, the complex may further comprise a fatty acid or a mixture of fatty acids (e.g. lauric acid, myristic acid, palmitic acid, caprylic acid, or a mixture comprising them, such as coconut oil), which may be present in an active to fatty acid weight ratio of about 3:1 to 1:3. In one embodiment, the biologically active compound is a cannabinoid and the complex further comprises a fatty acid or a mixture of fatty acids, which may be present in an active to fatty acid weight ratio of about 3:1 to 1:3.

Alternatively, complexes as herein described may be produced by other processes known for the preparation of complexes. For instance, these processes may include ball milling, extrusion, planetary mixing, etc.

III Complexes and Compositions

The carbohydrate polymer of the present disclosure forms a complex with at least one biologically active compound.

The carbohydrate polymers are suitable for pharmaceutical use and do not have a detrimental effect on the subject who will absorb the complex or a formulation thereof. Examples of carbohydrate polymers include glycosaminoglycans (e.g. hyaluronic acid (HA) or a salt thereof, such as HA having a Mw of between 0.3 and 1.2 M Da), cellulose, starch (amylose, amylopectin), chitin, chitosan, inulin, cyclodextrin, and the like, or a derivative or salt thereof, preferably hyaluronic acid or a salt thereof. The carbohydrate polymer may be further derived, crosslinked, or transformed before forming the complex with the biologically active molecule.

Suitable biologically active compounds include actives such as nutrients and nutraceuticals, natural products and extracts, small molecules drugs, and larger active molecules (biologics). For instance, the nutrient, nutraceutical, natural product or extract include, without limitation, amino acids, amino esters, amino alcohols, hydroxy acids, hydroxy esters, vitamins, turmeric (or curcumin), cannabinoids (e.g. tetrahydrocannabinol, cannabidiol, cannabinol, cannabigerol, cannabichromene, cannabicyclol, cannabivarin, tetrahydrocannabivarin, cannabidivarin, cannabichromevarin, cannabigerovarin, cannabigerolic acid, delta-tetrahydrocannabinolic acid, cannabidiolic acid, cannabichromenenic acid, cannabigerovarinic acid, tetrahydrocannabivarinic acid, cannabidivarinic acid, cannabichromevarinic acid, and the like). Examples of small molecule drugs (e.g. with a molecular weight below 900 g/mol) include antineoplastic agents (chemotherapy) and other oncology-related treatments (e.g. immunotherapies, adjuvants, etc.), antibiotics, antivirals, analgesics, anticoagulants, antidepressants, psychedelic therapy drugs (for example for OCD, PTSD, alcoholism, depression, cluster headaches, etc. including psilocybin, LSD, DMT, ketamine, etc.), antipsychotics, sedatives, anti-inflammatory agents, antidiabetics, cardiovascular agents, and the like. Finally, examples of larger active molecules (e.g. with a molecular weight above 900 g/mol) include nucleic acids, proteins and peptides (e.g. monoclonal antibodies, etc.).

In some examples, the complex may comprise additional ingredients, such as a stabilizer. In some examples, the complex may further comprise a fatty acid or a mixture of fatty acids, which may be present in an active to fatty acid weight ratio of about 3:1 to 1:3. In one embodiment, the biologically active compound is a cannabinoid and the complex further comprises a fatty acid or a mixture of fatty acids, which may be present in an active to fatty acid weight ratio of about 3:1 to 1:3. In other examples, the complex is formed in the presence of additional active compatible ingredients such that they become trapped and/or complexed within the carbohydrate polymer network.

The carbohydrate polymer of the present complex generally acts as a vehicle which may provide for an increased bioavailability of or sustained exposure to the active(s). Other improved properties may include enhanced solubility, reduced toxicity and side effects, an increased resistance to enzymatic degradation (stabilization effect), thus requiring less frequent reinjection, than the non-complexed compositions. In some cases, the present complex may allow the use of biologically active molecules in an oral formulation rather than an injectable solution. Other advantages may be dependent on the carbohydrate polymer used and on its properties. For instance, when the carbohydrate polymer is hyaluronic acid or a salt thereof (HA), a targeted delivery of the biologically active molecule may be achieved. Indeed, a HA-containing complex may be adapted for targeted delivery to cells and tissues known for expressing or overexpressing specific hyaluronic acid receptors such as CD44 and RHAMM. For instance, these are known to be present on most cancer cells.

The present disclosure also contemplates compositions suitable for administration to a subject (e.g. human or animal), and which comprise the complex as defined herein together with a pharmaceutically acceptable carrier. For instance, the composition comprises the present complex in a concentration of about 0.6% to about 10% w/w in the total composition. For instance, the concentrations of carbohydrate polymer and biologically active material are:
from 0.5 to 5.0% w/w of carbohydrate polymer, or from about 1.0% to about 2.0% w/w of carbohydrate polymer, or from about 1.0% to about 1.5% w/w of carbohydrate polymer; and
from 0.1 to 3.0% w/w, or from about 0.1% to about 2.0% w/w, or from about 0.1% to about 1.5%, or from about 0.1% to about 1.2%, or from 0.1% to about 0.5%, or from about 0.1% to about 0.4% w/w of biologically active compound(s);

wherein at least a portion of the biologically active compound(s) is complexed with the carbohydrate polymer.

In one example, the composition comprises only a single type of biologically active compound. Alternatively, the composition comprises a mixture of different types of biologically active compounds.

The present compositions also include, as the remainder of the composition, a pharmaceutically acceptable carrier. For instance, the pharmaceutically acceptable carrier may facilitate processing of the complex into pharmaceutically acceptable compositions. As used herein, the expressions “pharmacologically acceptable carrier” and “pharmacological carrier” equally refer to any carrier that has substantially no long term or permanent detrimental effect when administered to subjects including humans and encompasses expressions such as “pharmacologically acceptable vehicle, stabilizer, diluent, additive, auxiliary, or excipient.” A carrier is generally mixed with a complex or permitted to dilute or enclose the complex and is for example a solid, semi-solid, or liquid agent. It is understood that the complex is soluble or is delivered as a suspension in the desired carrier or diluent. In an embodiment of the present disclosure, the carrier or diluent includes water, saline or buffered saline, glycerol, propylene glycol, liquid polyethylene glycol, and the like.

The present compositions may also further comprise additional excipients, such as isotonic agents, stabilizers, antimicrobial agents, and other excipients commonly used in the field. In some examples, the composition comprises a stabilizer. Illustrative examples of stabilizers include L—or D-carnitine and glutathione. For instance, at least a portion of the one or more stabilizers may be complexed or crosslinked to the carbohydrate polymer. The compositions may thus comprise from about 0.1% to about 2.0% w/w, or from about 0.1% to about 1.5% w/w, or from about 0.1% to about 1.0% w/w of stabilizer.

The compositions of the present disclosure may further comprise a hydrophilic polymer. For instance, the compositions may comprise from about 0.5% to about 2.0% w/w, or from about 1.0% to about 2.0% w/w, or from about 1.2% to about 1.7% w/w of hydrophilic polymer. In some embodiments, the hydrophilic polymer is a second carbohydrate polymer such as carboxymethylcellulose.

Examples of compositions include tablets (e.g. hard-pressed or chewable tablets), capsules (e.g. gel capsules), lozenges, sprays, patches, syrups, liquid solutions, and the like.

Alternatively, the present compositions also include edible compositions such as food and beverages, including cookies, cakes and other pastries, puddings, chocolates, soft and hard candies (such as gummies and mints), beverages (such as water, juices, soft drinks, tea, dealcoholized wine or beer, etc.) and concentrated beverages, where the compositions comprise a complex of a carbohydrate polymer and a biologically active compound as defined herein. The composition will further contain a carrier selected for its suitability for the intended purpose, such as water or other aqueous based drinkable solutions like dealcoholized beer or wine, tea, natural or artificial juice, gelatin, dough, chocolate, etc. Additional ingredients may also be included such as preservatives, buffer agents, etc. In some preferred examples, the biologically active compound is a nutraceutical, cannabinoid or psychedelic therapy drug, for instance, the biologically active compound is a cannabinoid as defined herein.

IV. Uses and Methods of Use

The carbohydrate polymer of the present disclosure and at least one biologically active compound form a complex. As described herein, the complex and its compositions may have improved properties compared to the corresponding non-complexed biologically active compound. In an aspect, the present disclosure includes administering the composition to a subject in need thereof, e.g. having a medical condition. As used herein, the term “administering” refers to delivering a composition comprising a complex as defined herein to a subject and which administration potentially results in a clinically, therapeutically, or experimentally beneficial result.

The actual delivery mechanism and dosage regimen is readily determined by a person of ordinary skill in the art by taking into account factors, including, without limitation, the type of medical condition, the cause of the medical condition, the severity of the medical condition, the degree of relief desired, the duration of relief desired, the particular composition used, the pharmacodynamics of the particular composition used, the nature of the other compounds included in the particular composition used, the particular route of administration, the particular characteristics, history and risk factors of the individual, such as, e.g., age, weight, general health and the like, or any combination thereof. In an embodiment, the compositions of the present disclosure are administered to an individual by different modes, including oral, parenteral, nasal, mucosal, transdermal, intravascular (IV), intraarterial (IA), intramuscular (IM), and subcutaneous (SC) administration routes, preferably oral.

In some examples, the complex provides for sustained release of the biologically active compound, for instance, over a period of at least one day. The complex may thus provide for a controlled active release profile of the biologically active compound over time, e.g. the release being dependent on the enzymatic degradation of the carbohydrate polymer.

Uses of the present complexes are also contemplated for delivering the biologically active compound. Such delivery may be targeted to specific cells, tissues or organs depending on the carbohydrate polymer used in the complex. For instance, where the carbohydrate polymer is hyaluronic acid or a salt thereof, delivery the biologically active compound may be targeted to neoplastic cells, including fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, lymphomas, sarcomas and epithelial cancers, such as ovarian cancers and breast cancers.

Generally, contemplated are also uses of the present complexes and compositions in the treatment of neoplasm, bacterial infection, viral infection, pain, depression, sleep disorders, inflammation, diabetes, or as a cardiovascular agent, anticoagulant or antipsychotic agent. Also contemplated are diseases and disorders which could benefit from cannabinoid or psychedelic drug therapy. Further contemplated are uses for improving general health or simply for recreational purposes.

Alternatively, the present disclosure also relates to the use of the compositions and complexes in the preparation of food and beverages, as well as to the food and beverages as describes herein.

### Example

The following non-limiting example is an illustrative embodiment and should not be construed as further limiting the scope of the present invention.

### Example 1: Preparation of Complexes

All complexes were prepared by acoustic mixing under resonant conditions at moderate frequency (around 60 Hz) and forcing energy of between 50 g to 100 g, mainly at 100 g. The ingredients, including the carbohydrate polymer and active ingredient are placed into the glass vessel of the mixing apparatus. Mixing was carried out at 25° C. for between 1 and 10 minutes. Complexes prepared by the method are summarized in Table 1.

TABLE 1
Summary of Ingredients in complexes

| | Active | Carbohydrate | AI/excipient |
|---|---|---|---|
| Complex | ingredient (AI) | (excipient)* | wt. ratio |
| 1 | CBD | HA (0.40 MDa) | 1:4 |
| 2 | CBD | HA (0.40 MDa) | 1:9 |
| 3 | CBD | HA (1.06 MDa) | 1:4 |
| 4 | CBD | HA (1.06 MDa) | 1:9 |
| 5 | THC | HA (0.40 MDa) | 1:9 |
| 6 | THC | HA (1.06 MDa) | 1:9 |
| 7 | CBD | Chitosan | 1:9 |
| 8 | CBD | Starch | 1:4 |
| 9 | CBD | Carboxymethylcellulose | 1:8 |
| 10 | CBD | Inulin | 1:4 |
| 11 | CBD | B-cyclodetrin | 1:5 |
| 12 | Turmeric | HA (0.40 MDa) | 1:9 |
| 13 | Omega 3 | HA (1.06 MDa) | 1:2 |
| 14 | Raloxifene | HA (1.06 MDa) | 1:9 |
| 15 | Letrozole | HA (1.06 MDa) | 1:9 |

*HA: Hyaluronic acid as its sodium salt

An additional excipient such as fatty acids (e.g., coconut oil) were also added in a 1:1 or 1:2 weight ratio during the formation of Complexes 1 to 10. The fatty acid-active ingredient mixture may be briefly warmed up to 60° C. or 65° C. before mixing with the carbohydrate.

Other complexes are prepared using, for instance, curcumin (instead of trumeric) or magnesium ascorbyl phosphate and a carbohydrate polymer (like HA) in ratios between 1:4 and 1:9.

In the case of magnesium ascorbyl phosphate, the resulting complex (about 12 g) is subsequently mixed with sodium chloride (between 100 to 200 g) and phosphate buffer (pH 7.4; 100 g) and then reconstituted in distilled water up to a total volume of 10 L. Following degassing, the homogeneous solution is distributed in glass syringes (sizes ranging from 0.5 mL to 3.0 mL) or glass vials (sizes ranging from 0.5 mL to 20.0 mL). Finally, the syringes and/or vials are sterilized under reduced pressure at a temperature of 120° C. over a period of about 45 minutes.

### Example 2: Solubility Studies

Tests were carried to verify the solubility in water of the complexes prepared in Example 1. In each case, an amount of about 10-12 mg of complex was weighted. Distilled water (1 ml) was added, and the mixture was vortexed. If necessary, the mixture was heated for a few seconds in a water bath at about 60° C. Complexes 1 to 6, 9, 10 and 13 were shown to be fully soluble. Complexes 7, 8, 11, 12, 14 and 15 were not completely soluble in water at the tested concentration. These may still be water soluble at other concentrations.

### Example 3: Pharmacokinetic Profiles

(a) CBD and THC complexes pharmacokinetic profiles

Oral pharmacokinetic (PK) profile of CBD and THC formulations in HA, respectively complexes 3 and 5 (se Table 1 above), were conducted and compared with formulations without HA. Fasted male rats (2 or 3 per group) were administered per os with formulations to reach 20 mg/kg of either CBD or THC complexes alongside pure CBD or THC in coconut oil at a concentration of 4 mg/g of oil and at the same dosage.

The complexes were administered as a single gavage using solutions stirred at room temperature overnight prior to the day of administration. Coconut oil-based formulations (THC or CBD) were liquefied briefly by heating at 60° C. until being loadable in gavage syringes.

Blood samples were collected at 0, 0.25, 0.5, 1, 2, 4, 8, and 24 h. Plasma concentrations of active ingredient were determined by GC-MS/MS to determine the PK parameters. These were calculated using usual methods.

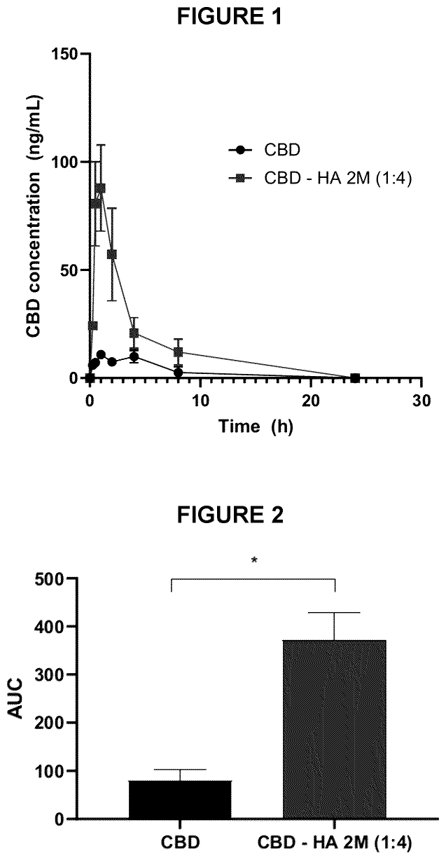

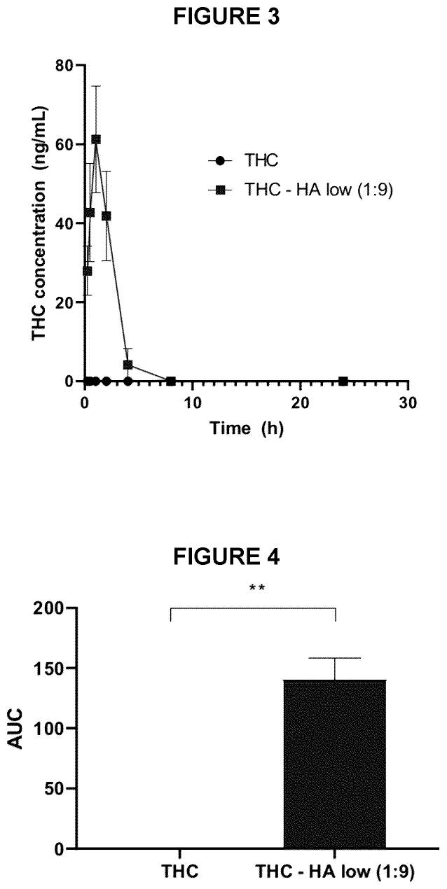

CBD plasma concentrations over time after administration are depicted in the graph of FIG. 1 while the AUC parameters are shown in FIG. 2. THC plasma concentrations are reported in FIG. 3 while the AUC parameters are presented in FIG. 4. A clear increase in plasma concentration of both CBD and THC can be observed for the HA complexes in comparison to each the active ingredient in coconut oil. For CBD, the Cₘₐₓ raised from an average of 12.7 ng/mL to 87.9 ng/mL, which was about 7-fold higher. For THC, average circulating levels raised to 64.6 ng/mL whereas they were mostly below the limit of quantitation when dosed with pure THC. The AUC could also not be calculated for pure THC (non-complexed) for the same reason.
(b) Letrozole Complex Pharmacokinetic Profile

The oral pharmacokinetic (PK) profile of a letrozole formulation in HA, complex 15 (se Table 1 above), was conducted and compared with that of a formulation without HA. The procedure followed was as described in (a) except that dosages of 2.5 mg/kg and 5 mg/kg were used for the letrozole in HA and of 2.5 mg/kg for the pure letrozole formulation.

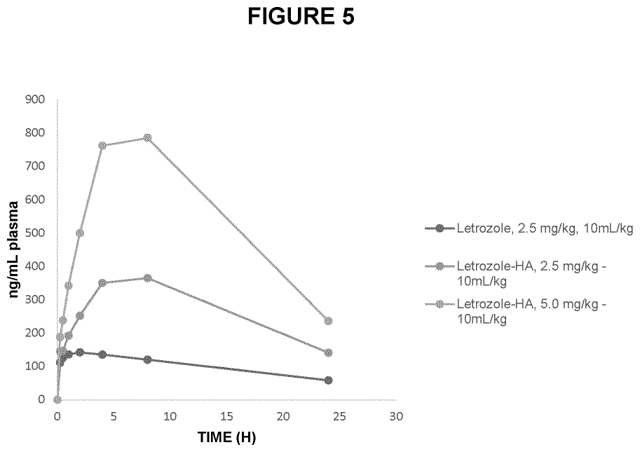

Plasma letrozole concentrations obtained are shown in FIG. 5. As can be seen, a clear increase in Cₘₐₓ could be observed with the HA-complexed letrozole compared to the non-complexed equivalent even at the same dosage.

Numerous modifications could be made to any of the embodiments described above without distancing from the scope of the present invention. Any references, patents or scientific literature documents referred to in the present application are incorporated herein by reference in their entirety for all purposes.

1. A process for preparing a complex of a carbohydrate polymer and at least one biologically active compound, the process comprising:
(a) mixing the carbohydrate polymer with the at least one biologically active compound to produce a mixture;
(b) feeding the mixture into a resonant acoustic mixer (RAM); and
(c) operating the RAM under resonant acoustic conditions, preferably at moderate frequency and forcing energy of between 50 g to 100 g, to produce the complex.

2. The process of claim 1, wherein step (c) is carried out at a temperature of between about 20° C. and about 40° C., or between about 25° C. and about 30° C.

3. The process of claim 1 or 2, wherein step (c) is carried out at a moderate frequency of about 20 Hz to about 90 Hz, preferably around 60 Hz.

4. The process of any one of claims 1 to 3, wherein step (c) comprises a residence time of about 10 second to about 10 minutes.

5. The process of claim 4, wherein the residence time is between about 30 seconds and 5 minutes.

6. A process for preparing a complex of a carbohydrate polymer and at least one biologically active compound, the process comprising:
a) adding the carbohydrate polymer and the at least one biologically active compound into a RAM; and
b) mixing the carbohydrate polymer and the at least one biologically active compound under resonant acoustic conditions at moderate frequency and forcing energy of between 50 g to 100 g, to produce the complex.

7. The process of claim 6, wherein step (b) is carried out at a temperature of between about 20° C. and about 40° C., or between about 25° C. and about 30° C.

8. The process of claim 6 or 7, wherein step (b) is carried out at a frequency of about 20 Hz to about 90 Hz, preferably around 60 Hz.

9. The process of any one of claims 6 to 8, wherein step (b) comprises a residence time of about 10 second to about 10 minutes.

10. The process of claim 9, wherein the residence time is between about 30 seconds and 5 minutes.

11. The process of any one of claims 1 to 10, wherein the carbohydrate polymer is selected from glycosaminoglycans (e.g. hyaluronic acid or a salt thereof), cellulose, starch (amylose, amylopectin), chitin, chitosan, inulin, cyclodextrin, and the like.

12. The process of claim 11, wherein the carbohydrate polymer is hyaluronic acid or a salt thereof.

13. The process of any one of claims 1 to 12, wherein the complex comprises from about 50 to about 95% (w/w) of the carbohydrate polymer.

14. The process of any one of claims 1 to 13, wherein the complex comprises from about 5 to about 50% (w/w) of the biologically active compound.

15. The process of any one of claims 1 to 14, wherein the weight ratio of biologically active compound to carbohydrate polymer is from 1:1 to 1:100, or from 1:4 to 1:9.

16. The process of any one of claims 1 to 15, wherein the biologically active compound is selected from amino acids, amino esters, amino alcohols, hydroxy acids, hydroxy esters, vitamins, and combinations thereof.

17. The process of claim 16, wherein the vitamin is selected from vitamin A, vitamin B1, vitamin B2, vitamin B₃, vitamin B₅, vitamin B₆, vitamin B9, vitamin B₁₂, vitamin C, vitamin D, vitamin E, vitamin H, vitamin K, and combinations thereof.

18. The process of any one of claims 1 to 15, wherein the biologically active compound is a pharmaceutical active ingredient selected from small molecule drugs, including antineoplastic agents, antibiotics, antivirals, analgesics, anticoagulants, antidepressants, psychedelic therapy drugs (for example for OCD, PTSD, alcoholism, depression, cluster headaches, etc. including psilocybin, LSD, DMT, ketamine, etc.), antipsychotics, sedatives, anti-inflammatory agents, antidiabetics, cardiovascular agents, and the like.

19. The process of any one of claims 1 to 18, wherein the biologically active compound has a molecular weight below 0.9 kDa.

20. The process of any one of claims 1 to 15, wherein the biologically active compound has a molecular weight of at least 0.9 kDa.

21. The process of claim 20, wherein the biologically active compound is a pharmaceutical active ingredient selected from nucleic acids, proteins and peptides.

22. The process of claim 21, wherein the biologically active compound is a monoclonal antibody.

23. The process of any one of claims 1 to 15, wherein the biologically active compound is a natural product or extract.

24. The process of claim 23, wherein the natural product or extract comprises turmeric (or curcumin) or a cannabinoid.

25. The process of claim 24, wherein the natural product or extract comprises a cannabinoid selected from tetrahydrocannabinol, cannabidiol, cannabinol, cannabigerol, cannabichromene, cannabicyclol, cannabivarin, tetrahydrocannabivarin, cannabidivarin, cannabichromevarin, cannabigerovarin, cannabigerolic acid, delta-tetrahydrocannabinolic acid, cannabidiolic acid, cannabichromenenic acid, cannabigerovarinic acid, tetrahydrocannabivarinic acid, cannabidivarinic acid, cannabichromevarinic acid, and the like.

26. A complex prepared by a process as defined in any one of claims 1 to 25.

27. A complex of a carbohydrate polymer and at least one biologically active compound.

28. The complex of claim 27, wherein said carbohydrate polymer is as defined in claim 11 or 12.

29. The complex of claim 27 or 28, wherein said biologically active compound is as defined in any one of claims 16 to 25.

30. The complex of any one of claims 27 to 29, wherein the complex comprises from about 50 to about 95% (w/w) of the carbohydrate polymer.

31. The complex of any one of claims 27 to 30, wherein the complex comprises from about 5 to about 50% (w/w) of the biologically active compound.

32. The complex of any one of claims 27 to 31, wherein the weight ratio of biologically active compound to carbohydrate polymer is from 1:1 to 1:100, or from 1:4 to 1:9.

33. A composition comprising the complex of any one of claims 26 to 32 together with a carrier.

34. The composition of claim 33, further comprising a stabilizer.

35. The composition of claim 33 or 34, said composition being a solid composition (e.g. powder composition).

36. The composition of claim 33 or 34, said composition being a liquid composition.

37. The composition of any one of claims 33 to 36, wherein the concentration of the complex in the composition is from about 0.6 to about 10% (w/w).

38. The composition of any one of claims 33 to 37, wherein said composition is a pharmaceutical composition for use as a medication.

39. The composition of claim 38, said pharmaceutical composition being in the form of a tablet (e.g. hard-pressed or chewable tablet) or gel capsule.

40. An edible composition comprising the complex of any one of claims 26 to 32 together with a carrier.

41. The edible composition of claim 40, wherein said carrier is selected from water or other aqueous based drinkable solutions like dealcoholized beer or wine, tea, natural or artificial juice, gelatin, dough, chocolate, etc.

42. The edible composition of claim 40 or 41, wherein said edible composition is a solid or semi-solid food composition.

43. The edible composition of claim 42, wherein said edible composition is selected from cookies, cakes and other pastries, puddings, chocolates, soft and hard candies (such as gummies and mints).

44. The edible composition of claim 40 or 41, wherein said edible composition is a liquid composition.

45. The edible composition of claim 44, wherein said edible composition is a beverage (such as water, juices, soft drinks, tea, dealcoholized wine or beer, etc.) or concentrated beverage.

46. The edible composition of any one of claims 40 to 45, wherein the biologically active compound is selected from a natural product or extract comprising a nutraceutical or a cannabinoid, and a psychedelic drug (e.g. psilocybin, LSD, DMT, ketamine, etc.).

47. The edible composition of claim 46, wherein the natural product or extract comprises a cannabinoid.

48. The edible composition of claim 47, wherein the cannabinoid is selected from tetrahydrocannabinol, cannabidiol, cannabinol, cannabigerol, cannabichromene, cannabicyclol, cannabivarin, tetrahydrocannabivarin, cannabidivarin, cannabichromevarin, cannabigerovarin, cannabigerolic acid, delta-tetrahydrocannabinolic acid, cannabidiolic acid, cannabichromenenic acid, cannabigerovarinic acid, tetrahydrocannabivarinic acid, cannabidivarinic acid, cannabichromevarinic acid, and the like.

49. The edible composition of any one of claims 40 to 48, wherein the carbohydrate polymer is a glycosaminoglycan (e.g. hyaluronic acid or a salt thereof).

50. Use of a complex as defined in any one of claims 26 to 32 or of a composition as defined in any one of claims 33 to 39, in the treatment of neoplasm, bacterial infection, viral infection, pain, depression, sleep disorders, inflammation, diabetes, as a cardiovascular agent, anticoagulant or antipsychotic agent, or of diseases and disorders which could benefit from cannabinoid or psychedelic drug therapy.
